(19) United States
(12) Patent Application Publication
(10) Pub. No.: US 2023/0365922 A1
(43) Pub. Date: Nov. 16, 2023

(54) Platelet Generation

(71) Applicant: The University of Bristol, Bristol (GB)

(72) Inventors: Alistair William Poole, Bristol (GB); Xioajuan Zhao, Bristol (GB)

(21) Appl. No.: 18/245,560

(22) PCT Filed: Sep. 17, 2021
(86) PCT No.: PCT/EP2021/075704

§ 371 Date: Mar. 16, 2023

(30) Foreign Application Priority Data
Sep. 18, 2020 (GB) 2014727.8

(51) Int. Cl. C12N 5/00; C12N 5/078; C12M 1/12

(52) U.S. Cl. CPC C12N 5/0068; C12M 25/14; C12N 5/0644

(57) ABSTRACT

The invention provides a method for generating platelets or platelet-like particles comprising passing a source material comprising one or more cells through a micro-passage to produce a passaged material comprising platelets or platelet-like particles. The invention also provides platelets and platelet-like particles produced using the method and uses thereof.

### FIELD OF THE INVENTION

The invention relates to methods for generating platelets and platelet-like particles, to platelets generated using the methods, to apparatus for use in platelet generation, and to uses of the platelets.

### BACKGROUND TO THE INVENTION

Platelets are the smallest cellular component of blood, with critical roles in haemostasis, thrombosis, inflammation, vascularization and tissue repair. They are generated from large polyploid cells, megakaryocytes, although the location of their generation is currently not fully clear. Although the bone marrow provides a major space for maturation of hematopoietic stem cells to all blood cell lineages, including megakaryocytes, much of the platelet generation in the body may happen at sites distant to the marrow. Whole megakaryocytes, or large fragments of these cells, have been observed to exit the marrow into the circulation (Brown et al. 2018), whereupon their next microcirculation destination is the lung, which has recently been shown to be a site for the generation of substantial numbers of platelets (Lefrancais et al. 2017).

Current models of platelet generation are based on the premise that platelets are generated from bone marrow sinusoids. The prevailing view is that, in the body, megakaryocytes squeeze out of the bone marrow and, as part of a megakaryocyte enters the blood, platelets are sheared off by the force of the fast moving blood flow. Known devices for producing platelets, such as that described by Thon, et al., Blood, 18 Sep. 2014, Vol. 124, Number 12, mimic this by having a microporous membrane with MK cells squeezing through into a high flow liquid. However, the results of such systems are poor, with only a few platelets being formed per MK cell and the platelets having poor functionality.

The inventors believe that the current understanding of platelet formation is incorrect. They have developed improved methods of platelet generation, which result in both increased numbers of platelets being formed per MK cell and increased functionality of platelets generated.

Rather than using shear force to break up MK cells, the inventors have developed methods using compression force, particularly repeated compression force, applied to MK cells, resulting in platelet production.

### SUMMARY OF THE INVENTION

The invention provides a method for generating platelets or platelet-like particles comprising the step of:

a) passing a source material comprising one or more cells through a micro-passage to produce a passaged material, the passaged material preferably comprising platelets or platelet-like particles.

A platelet or platelet-like particle is a blood component, the function of which is to react to bleeding, initiating a blood clot. The term platelet is well known in the art. A platelet-like particle is a particle having some or all of the features of a platelet, such as the ability to adhere to surfaces, other cells, other platelets or platelet-like particles, solid phase substances, the ability to release contents of granules upon activation, the ability to release extracellular vesicles, including microparticles and exosomes, the ability to be procoagulant, including the surface expression of negatively charged phospholipids such as phosphatidylserine and/or the ability to change shape rapidly.

The source material comprises one or more cells such as stem cells, particularly iPSCs; intermediate and/or final products of stem cell differentiation such as hemogenic endothelia, hematopoietic progenitor cells, megakaryocytes, endothelial cells, leukocytes, erythrocytes bone marrow cells, blood cells, lung cells and cells comprising basement membranes. In particular, it comprises one or more megakaryocytes or precursors thereof. The step of passing the source material through the micro-passage particularly comprises or consists of passing one or more, especially a plurality of cells, through one or more micro-passages, especially through a plurality of micro-passages.

The cells may be from any appropriate origin. For example, the cells may be mammalian, especially human or rodent, particularly murine. They may be native or generated ex vivo. Where the cells are native, they may be isolated cells.

The cells may be wild type or may be engineered or otherwise manipulated to include one or more desired characteristics, particularly characteristics that may also be found in the platelets generated. For example, the cells may be engineered to have enhanced therapeutic properties, including, for example enhanced release of tissue repair genes, proteins and polypeptides or other factors, modulated thrombotic activity, enhanced homing to particular sites. This may be particularly useful in generating platelets and platelet-like particles that are especially useful for treating conditions such as cancer, metabolic disorders and complications thereof, and ischemic diseases including coronary artery disease and stroke.

The source material may also comprise CCL5, CXCL12, CXCL1O, SDF-1, FGF-4, SIPRI, RGDS, Methylcellulose, collagen, fibrinectin, fibrinogen, laminin, Matrigel, Flt-3, thrombopoetin (TPO), VEGF, PLL, IL3, IL6, IL9, IL1b, vitronectin, stem cell factor (SCF), or combinations thereof.

A megakaryocyte is a large cell, generally found in bone marrow. The term is well known in the art. Mature megakaryocytes generally express the protein CD41. Megakaryocytes used in the method of the invention may be native in origin or may be generated ex vivo, e.g. via the use of stem cells, such as iPSCs. In some embodiments, the megakaryocytes may be genetically engineered, for example to express or delete particularly surface proteins, such as CD41, or to enhance therapeutic potential of platelets produced.

Precursors of megakaryocytes are any cells that can, particularly that are programmed to, differentiate to produce megakaryocytes. For example, they may be stem cells, such as iPSCs or umbilical cord stem cells, optionally haemopoetic stem cells (CD34+ stem cells). The cells may be forward-programmed, and may require factors such as TPO and SCF to induce differentiation. In the latter case, such factors may be included in the source material.

The source material preferably comprises a medium. The medium may be selected according to the cells in the source material, for example depending on the origin of the cells and on whether and how they are programmed to differentiate.

In an embodiment, the megakaryocytes or precursors thereof are isolated from blood or bone marrow or are artificially generated.

The source material is generally passed from a first region, through the micro-passage to a second region. The method may comprise the step of applying pressure to the source material in the first to drive the source material to the second region. The second region may comprise a medium or other material into which the passaged material is received. Material in the second region, whether a medium or the like into which passaged material is received, or the passaged material itself, is generally relatively static. Where there is a flow in the material or medium in the second region, it is generally in the same direction of travel as the direction of travel of the source material through the micro-passage. The source material is generally not subject to shear force, particularly in the second region. Rather, the source material is subject to compression force, particularly repeated compression force as it passes through the micro-passages.

More than one micro-passage may be provided between the first region and the second region. Generally the method may comprise passing the source material through a plurality of micro-passages. The micro-passages may be provided in parallel or in series or both.

The micro-passage is a pathway along which the MK is passed. For example, in an embodiment, the micro-passage may be a channel, aperture or pore. The micro-passage may have walls that define the micro-passage, that is to say that define an opening through which the source material may pass. At any point along the micro-passage, the walls may surround the opening entirely, or may just be provided around part of the opening. The walls may be connected to each other, or may simply be positioned near each other to define the opening.

The micro-passage may be generally tubular in shape, having one generally circular wall. In that embodiment, the source material passes along the lumen of the tube.

The micro-passage may be irregular in shape.

Where multiple micro-passages are provided, the micro-passages may be the same or different shapes.

The length of that pathway, i.e. the distance from one end of the micro-passage to the other may vary, but is generally less than 5 mm. The pathway may be generally straight or may include one or more bends or changes of direction. When moving through the micro-passage, i.e. along the pathway, a cell in the source material is passed through at least one region in which the width and/or height of the micro-passage is restricted in size. Accordingly, at least one of the width and height of the micro-passage is preferably between 1 and 250 µm, between 1 and 200 µm, between 1 and 150 µm, between 1 and 100 µm, between 1 and 50 µm, between 1 and 25 µm, between 1 and 20 µm, between 1 and 15 µm, between 1 and 12.5 µm, between 1 and 10 µm, or between 1 and 5 µm at a point along its length. For example, the height of the micro-passage is preferably less than 250 µm, more preferably less than 200 µm, optionally less than 150 µm, optionally less than 100 µm, optionally less than 75 µm, optionally less than 50 µm, optionally less than 25 µm, optionally less than 20 µm, optionally less than 15 µm, optionally less than 12.5 µm, optionally less than 10 µm, optionally less than 5 µm at at least one point along the length of the micro-passage. Alternatively, or additionally, the width of the micro-passage is preferably less than 250 µm, more preferably less than 200 µm, more preferably less than 150 µm at at least one point along the length of the micro-passage. Alternatively or additionally, the height of the micro-passage is preferably less than 250 µm, more preferably less than 200 µm, optionally less than 150 µm, optionally less than 100 µm, optionally less than 75 µm, optionally less than 50 µm, optionally less than 25 µm, optionally less than 20 µm, optionally less than 15 µm, optionally less than 12.5 µm, optionally less than 10 µm, optionally less than 5 µm at at least one point along the length of the micro-passage. Alternatively, or additionally, the width of the micro-passage is preferably more than 1 µm, more preferably more than 5 µm, more preferably more than 10 µm at at least one point along the length of the micro-passage. In each micro-passage, the width and height may be the same or different. Where multiple micro-passages are provided, they may be the same or different sizes.

The terms width and height refer to dimensions of the micro-passage that are generally found in a plane that is generally perpendicular to the direction of travel of the cell through the micro-passage.

In one embodiment, the method comprises passing the material through a micro-passage more than once. For example, the passaged material produced after a first pass through the micro-passage may be passed through the micro-passage again, or may be passed through another micro-passage.

In that embodiment, the method may comprise repeating the step of passing the passaged material through a micro-passage. The method may comprise repeating the step at least once, twice, three, four, five, six, seven, eight, nine, ten, fifteen, twenty, twenty five or thirty or more times.

In one embodiment, the step of passing a source material comprising cells through a micro-passage comprises the following steps:
a) providing a source material in a first vessel;
b) passing the source material from the first vessel into a second vessel, via one or more micro-passages to produce a passaged material.

The source material is preferably passed via multiple micro-passages.

In that embodiment, the method may further comprise the step of

c) passing the passaged material from the second vessel into the first vessel, via one or more micro-passages.

The method may comprise repeating steps b) and c).

In one embodiment, the invention provides a method for producing platelets or platelet-like particles, comprising passing a megakaryocyte through a micro-passage having a width between 1 and 200 µm, the width of the micro-passage being generally perpendicular to the direction of travel of the megakayrocyte through the micro-passage.

The method may further comprise the step of analysing the passaged material for the presence of platelet or platelet-like particles. The analysis step may follow step b) or c), or a repeat of step b) or c), or it may be continuous, for example real-time analysis. The analysis may comprise sorting and/or counting particles by size.

The method may further comprise the step of extracting platelets or platelet-like particles from the passaged material. The step of extracting platelets or platelet-like particles may comprise filtering the passaged material to remove the platelets or platelet-like particles.

The method may further comprise applying pressure to the first vessel, or to the second vessel, to drive the material from one vessel to the other.

The method may further comprise oxygenating the source material, or the passaged material, or both.

In one embodiment, the step of passing the source material or the passaged material through a micro-passage comprises or consists of the step of passing the material through an ECMO (Extra Corporeal Membrane Oxygenation) machine, artificial lung or equivalent device. The ECMO machine comprises a membrane, a parallel array of channels or a hollow fibre oxygenator. In a preferred embodiment, the ECMO machine comprises a parallel array of channels or a hollow fibre oxygenator.

In another embodiment, the step of passing the source material or the passaged material through a micro-passage comprises or consists of the step of passing the material through a flow cell, particularly a high pressure flow cell.

In one embodiment, the step of passing the source material or the passaged material through a micro-passage comprises or consists of the step of passing the material through a barrier comprising multiple microchannels, for example a microchannel plate.

In one embodiment, the step of passing the source material or the passaged material through a micro-passage comprises or consists of the step of passing the material through a a microfluidic chip..

In one embodiment, the step of passing the source material or the passaged material through a micro-passage comprises or consists of the step of passing the material through a mesh filter or sieve.

In one embodiment, the step of passing the source material or the passaged material through a micro-passage comprises or consists of the step of passing the material through a tubular array.

A tubular array is preferably a collection of elongated tubes, for example of around or less than 200 µm in diameter. The tubes may be the same or different diameters. The tubes are preferably arranged together so that they are generally parallel. The tubes are positioned such that there are varying spaces between them, creating multiple passages through which the source material or passaged material may be passed. The tubes may be solid or hollow. They may be porous or not. When the tubes are hollow, the source material may also or alternatively be passed through a lumen of one or more tubes. The tubes may be made of any appropriate material, such as a polymer.

In one embodiment, the step of passing the source material or the passaged material through a micro-passage comprises or consists of the step of passing the material through a needle or plurality of needles.

In an embodiment, the micro-passages are found between or defined by tubes arranged in a generally parallel array.

Also provided is an apparatus for producing platelets, the apparatus comprising a first region for containing source material and a second region for receiving a passaged material, the first and second regions being connected by at least one micro-passage.

The apparatus may also comprise a means for driving the source material from the first region to the second region.

The first and second regions may, for example, each comprise or be formed by a vessel, chamber. They may be the same or different.

The micro-passage may be as described in relation to the method for generating platelets described herein. The first and second regions are generally connected by a plurality of micro-passage, for example at least 100, at least 200, at least 300, at least 400, at least 500, at least 1,000, at least 5,000, at least 10,000, at least 100,000, at least 250,000, at least 500,000, or around or at least 1,000,000 micro-passages. Said micro-passages may be found in parallel.

In one embodiment, the micro-passages may be found in a barrier or membrane, positioned between the first and second regions. The barrier or membrane may be a rigid barrier, such as a plate, especially a ceramic or glass plate. Alternatively, the barrier may be a flexible or mesh. Where it is a mesh, it may be, for example, a fibrous mesh, especially made of polymer fibres.

In another embodiment, the micro-passages may be found in a tubular array as described previously.

The apparatus may further comprise a means for returning the passaged material from the second region to the first region. The material may be returned to from the second region to the first region via the micro-passages, or without going through the micro-passages.

The apparatus may further comprise means for counting cells or particles and/or sorting cells or particles by size. It may further comprise means for filtering or otherwise removing platelets or platelet-like particles, or cells or other contaminants, from the passaged material. The filtration means may comprise, for example a hollow fibre filtration system, particularly a tangential flow filter. In some embodiments, the filtration means may itself comprise micro-passages, optionally allowing a further step of passing the material through a micro-passage as part of the filtration step.

Also provided are platelets or platelet like particles, obtainable or obtained using the method of the invention. The platelets or platelet like particles are preferably in a medium. The platelets or platelet-like particles are generally as previously defined. In an embodiment, the platelets or platelet-like particles a substantially synchronised, that is to say they are generally homogenous in terms of age, i.e. they were generated within about one to two hours of each other and show similar levels of degradation. Synchronicity of platelets and platelet-like particles is understood in the art. It can be measured by standard techniques, such as staining with acridine orange

In an embodiment, the platelets or platelet-like particles are substantially uniform in shape and/or size. For example, in an embodiment the smallest platelets or platelet-like particles in the medium are at least 70%, 75%, 80%, 85% of the size, for example of the diameter of the largest platelets or platelet-like particles in the medium. In that, or another embodiment, at least 70%, 75%, 80%, 85%, 90% or 95% of the platelets in the medium are between about or exactly 3.5 and 6 µm, 4 and 6 µm, 4 and 5.5 µm. in diameter.

In an embodiment, the platelets or platelet-like particles are generally the same shape, and have the shape of unactivated platelets. In particular, the platelets or platelet-like particles are generally biconvex discoid or lens shaped in shape. The platelets or platelet-like particles may have a microtubule ring.

In an embodiment, the platelets or platelet-like particles may have a reduced ratio of surface protein to internal protein, when compared to naturally occurring platelets. For example, the platelets or platelet-like particles may have reduced CD41 expression, when compared to naturally occurring platelets. For example, the platelets or platelet-like particles may have less than 80%, 75%, 70%, 65%, 60%, 55%, 50%, 45%, 40%, 35%, 30%, 25% of the CD41 expression seen on native platelets. The platelet or platelet-like particles may have, for example, less than 50×10³ CD41 per platelet or particle, or less than 45×10³ CD41, or less than 40×10³ CD41, or less than 35×10³ CD41, or less than 30×10³ CD41, or less than 25×10³ CD41. In another example, the platelets or platelet-like particles may have less CD42B or GPVI than native platelets.

In an embodiment, the platelets are HLA-nul or have reduced HLA.

The platelet or platelet-like particles may generally have characteristics found in the cells from which they are produced. For example, they may be generated from cells engineered to have enhanced therapeutic properties, including, for example enhanced release of tissue repair genes, proteins and polypeptides, modulated thrombotic activity, enhanced homing to particular sites.

Also provided is a pharmaceutical composition comprising the platelets or platelet-like particles according to the invention or extracts therefrom.

Also provided is a method of treating a subject in need of platelets or platelet-like particles comprising the step of administering the platelets or platelet-like particles according to the invention or one or more extracts therefrom to the subject.

Extracts from platelets or platelet-like particles are known in the art. They may include exosomes, particularly purified exosomes.

The step of administering platelets or platelet-like particles or extracts may comprise administering platelets, platelet extracts, platelet-rich plasma, platelet releasates, and platelet-like particles.

A subject in need of platelets or platelet-like particles, or extracts therefrom, may be a subject in conditions of thrombocytopenia, for example after traumatic injury, during major surgery, during or following chemotherapy. Alternatively, or additionally, the subject may have a naturally occurring low platelet count condition, such as ITP (immune-mediated thrombocytopenia) or aplastic marrow disease.

Platelets or platelet-like particles according to the invention or extracts therefrom may also be administered to enhance tissue repair and / or tissue vascularity. Preparations comprising the platelets or platelet-like particles according to the invention or extracts therefrom are rich in growth factors, angiogenic factors (stimulating new blood vessel growth) and microRNAs that regulate a variety of activities in the body including cell proliferation. They may be used in, for example, orthopedic settings, dental extractions and the treatment of wounds

Further provided is a platelet or platelet-like particle according to the invention or an extract therefrom, or a medium comprising platelets or platelet-like particles according to the invention, for use in therapy, especially in the treatment of thrombocytopenia or low platelet conditions or to enhance tissue repair or vascularity, as discussed in relation to the method of treatment of the invention.

The invention will now be described in detail, by way of example only, with reference to the drawings.

### DESCRIPTION OF THE DRAWINGS

FIG. 1 shows in vitro education of megakaryocytes to generate platelets using the lung model system, including 1. Deformation, 2. Enucleation and 3. Platelet abscission. FIG. 2 Mouse platelets generated per megakaryocyte by multiple passages through mouse ex vivo heart-lung vasculature with air ventilation.

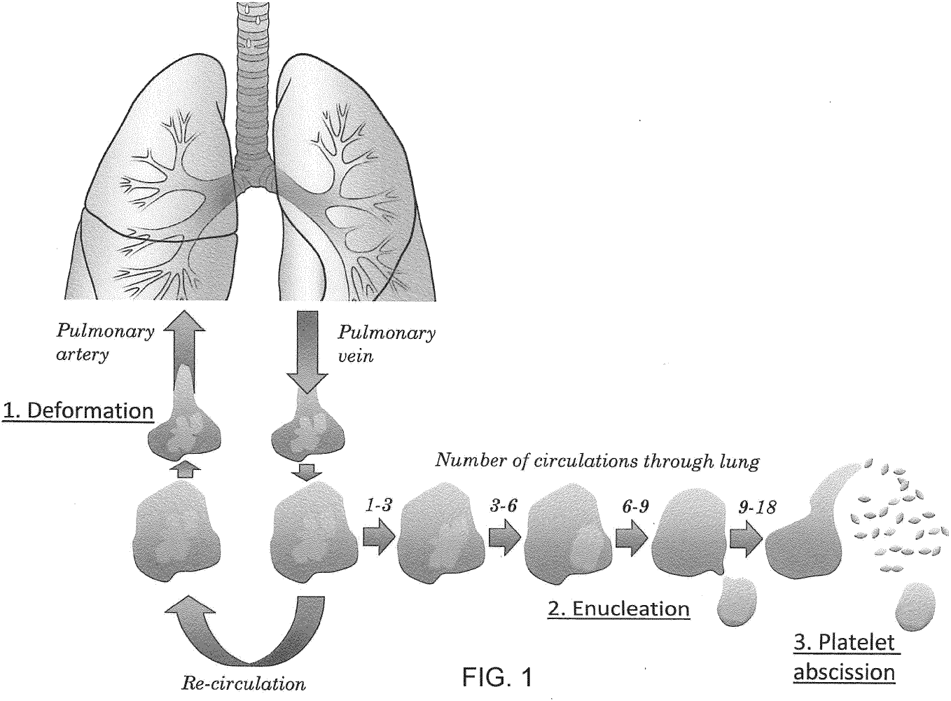

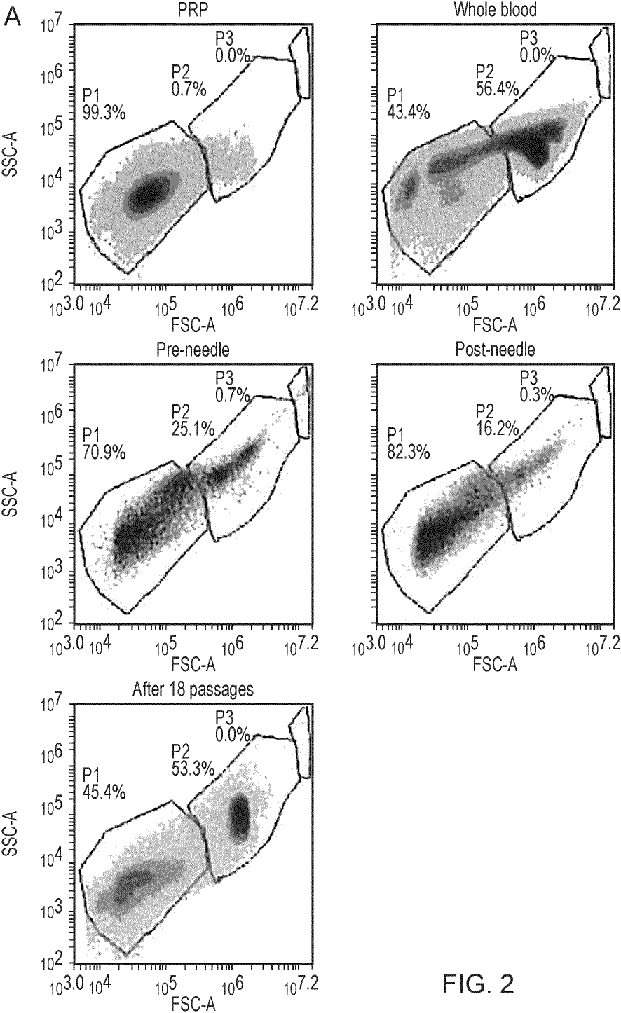

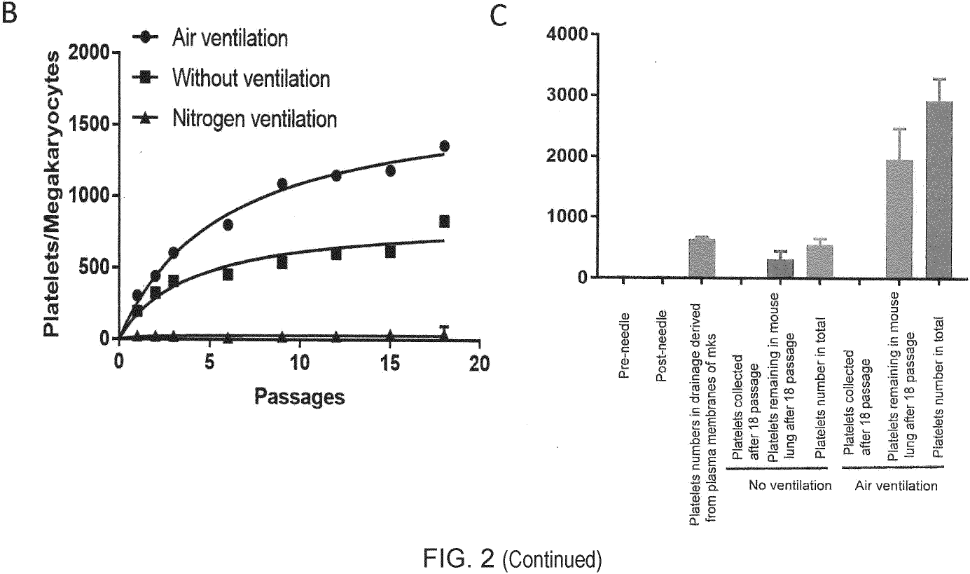

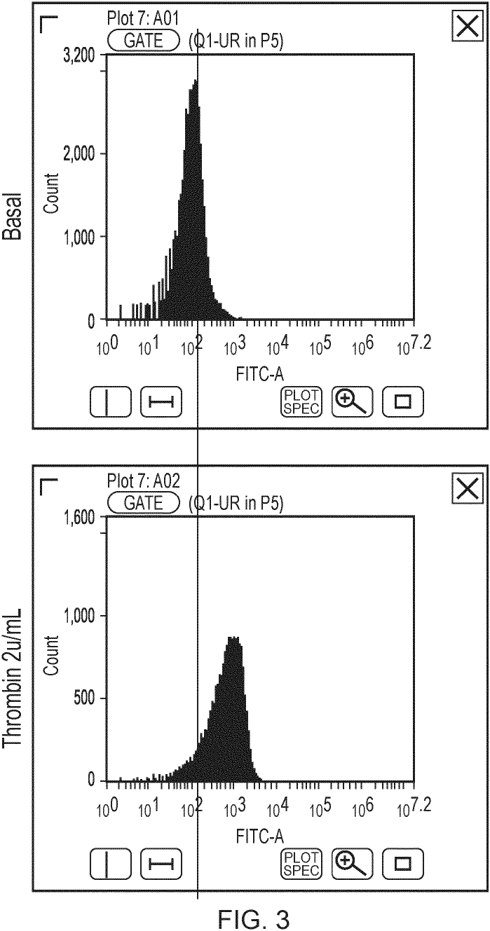

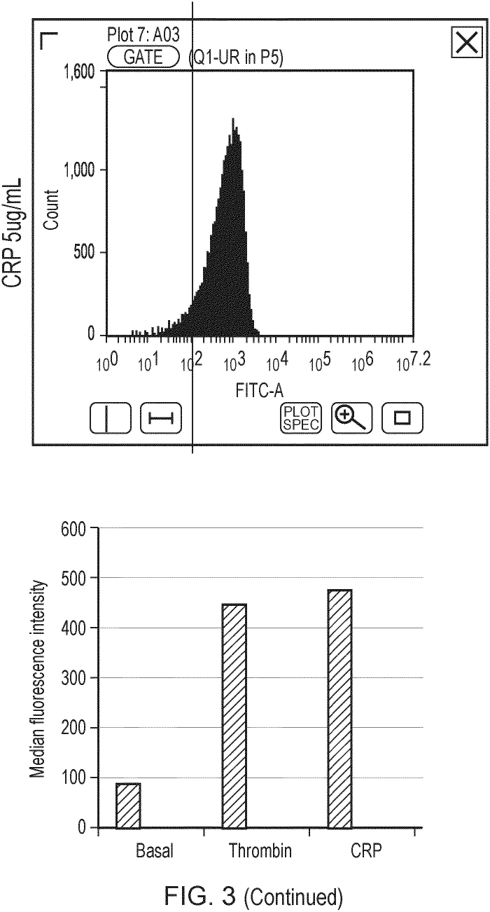

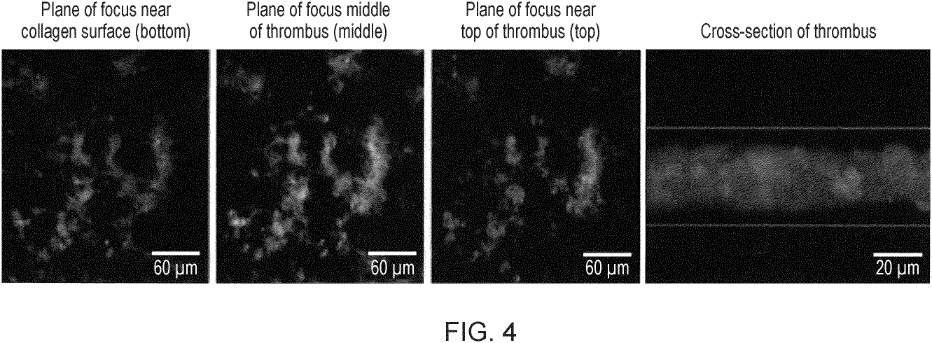

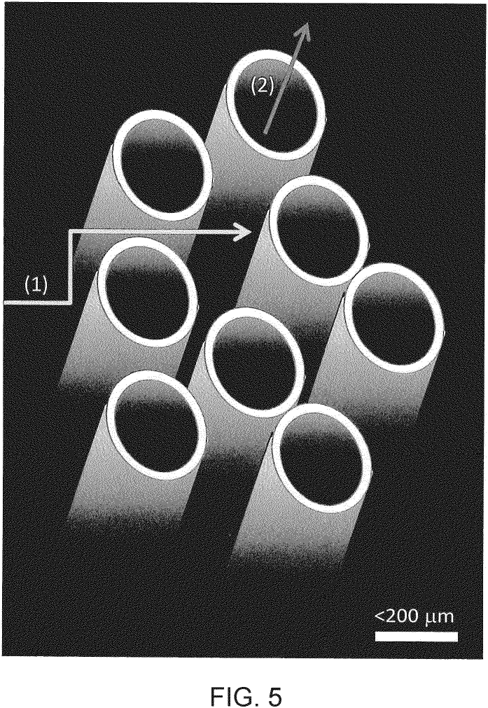

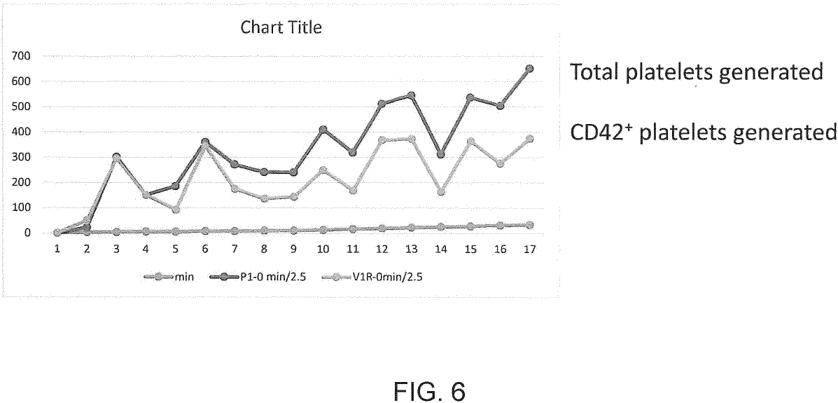

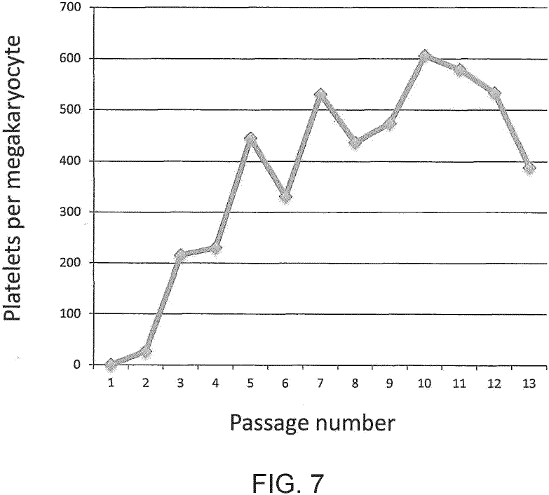

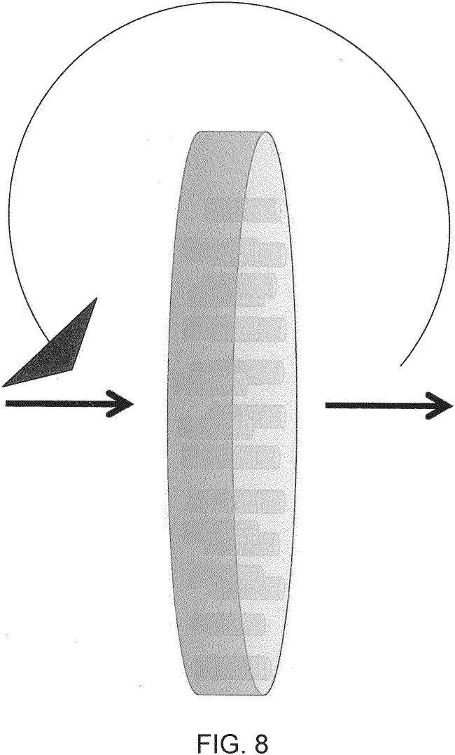

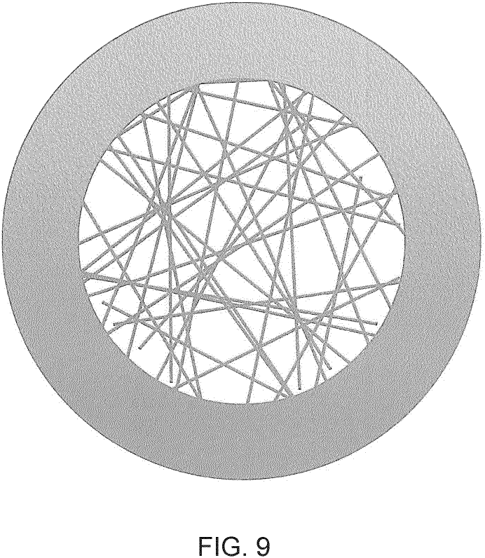

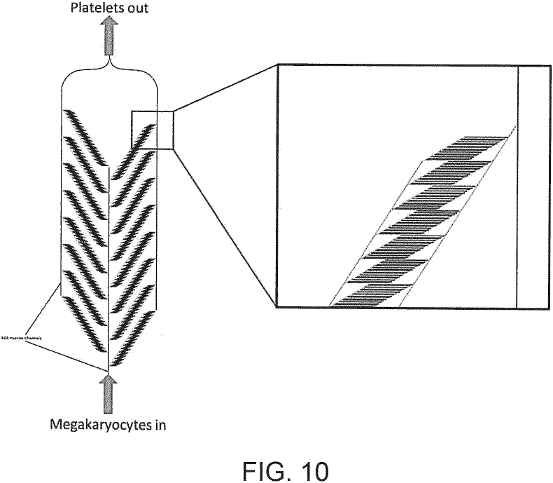

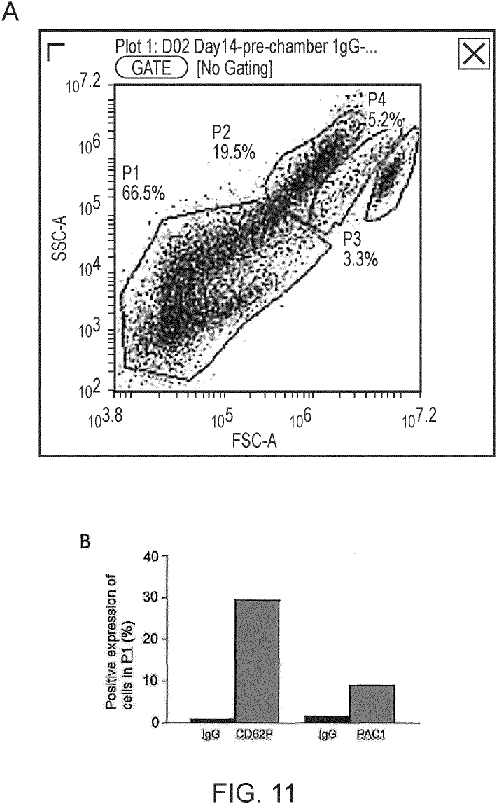

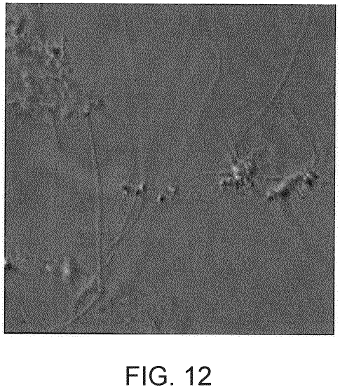

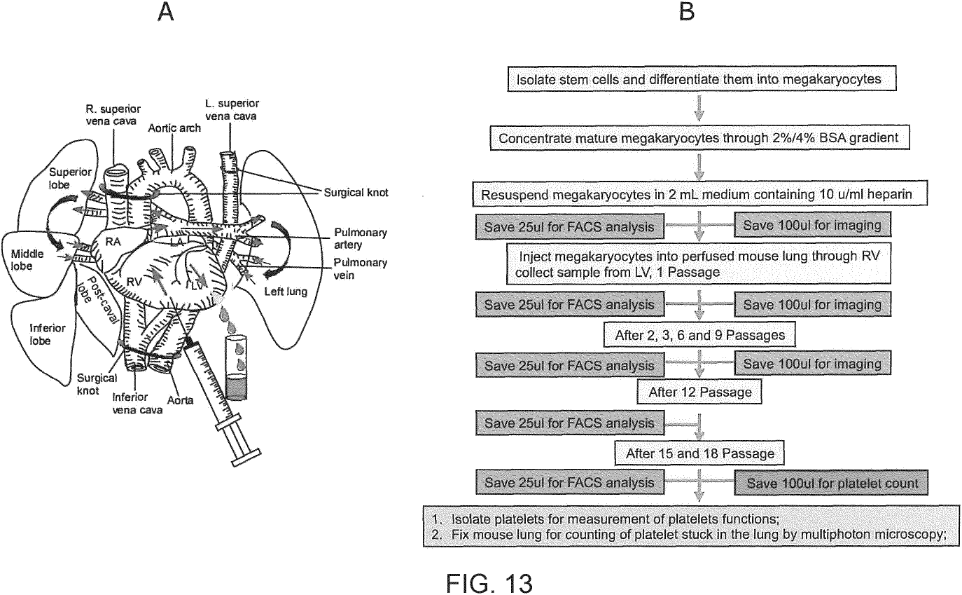

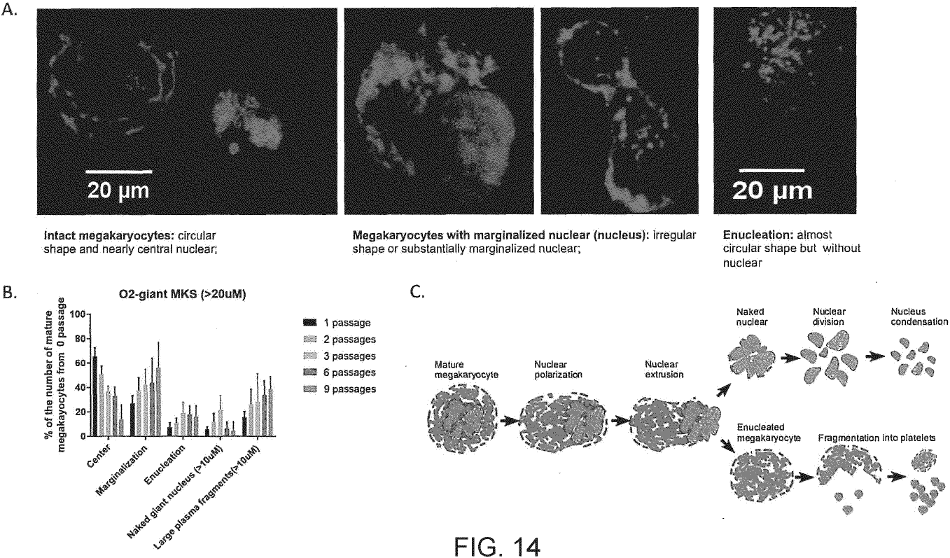

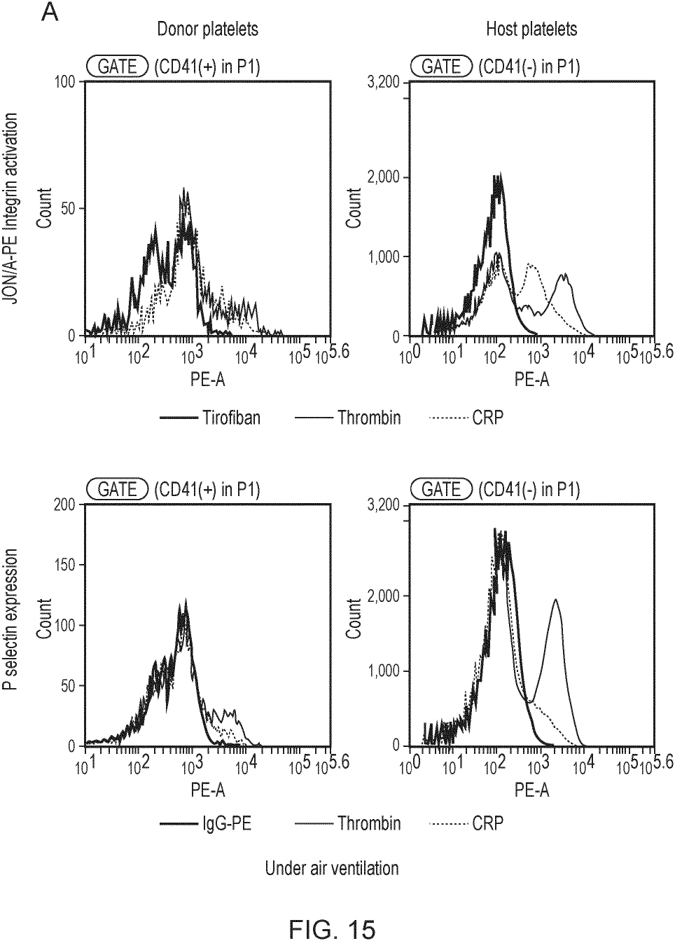

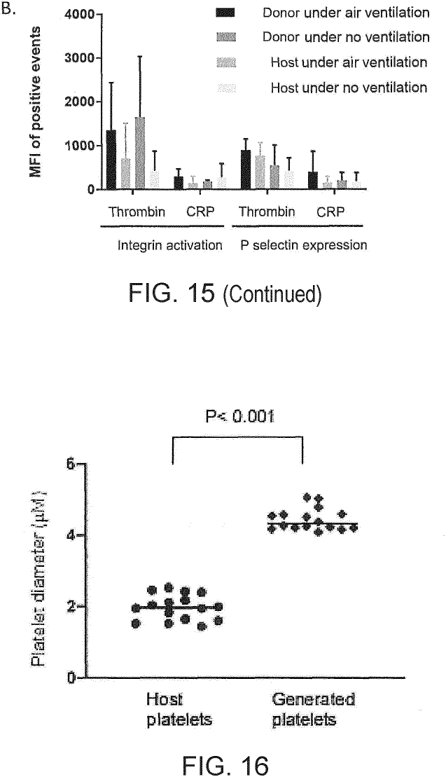

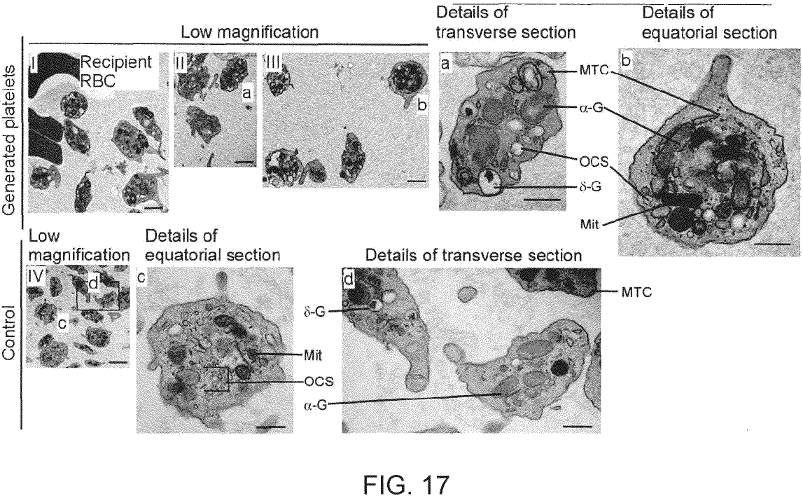

(A) FACS gating strategy for quantification of generated platelets. Mouse platelet-rich plasma was used to gate generated platelets (P1) at the FSC/SSC density plot upon size and complexity. Mouse whole blood was introduced to quantify CD41(+) events with similar size to erythrocytes and leucocytes (P2). Megakaryocyte resuspension of pre- or post-needle were evaluated to exclude whether the needle could break megakaryocytes into small pieces of cytoplasmic fragments. The numbers of generated platelets were determined by the CD41(+) events in P1 of eluate collected after 18^{th} passages.

(B) The numbers of generated platelets per megakaryocyte in eluates over different passages under air or nitrogen ventilation or without ventilation were measured by FACS and presented as line graphs.

(C) The numbers of generated platelets per megakaryocyte in both eluate and remaining in mouse lung under air ventilation and without ventilation were calculated and displayed as bar graphs.

FIG. 3 shows Thrombin and collagen-related peptide activate integrin a_{IIb}b₃ in synthesised mouse platelets.

FIG. 4 shows generated mouse platelets incorporate into growing thrombus in vitro, alongside endogenous platelets.

FIG. 5 shows an example of a tubular array, including showing the direction of passage of source material comprising cells through the array.

FIGS. 6 and 7 show the results of using the tubular array of FIG. 5 with mouse MKs and human cord blood derived MKs.

FIG. 8 shows an example of a barrier used in the apparatus, the barrier having multiple micro-passages, specifically microchannels.

FIG. 9 shows a further example of a barrier used in the apparatus, the barrier comprising a mesh creating micro-passages.

FIG. 10 shows a microfluidic system for use in the invention.

FIGS. 11 and 12 show the results of passaging human CD34+ and human iPSC derived MK cells through the system shown in FIG. 10.

FIG. 13 shows (A) injection of megakaryocytes into a perfused mouse lung, flow through the lung and collection; and (B) a flow chart of the steps taken.

FIG. 14 shows functionality in both the labeled (donor-derived) and unlabeled (host-derived) platelets.

FIG. 15 shows enucleation of MKs in the vasculature.

FIG. 16 shows sizes of platelets obtained using the method of the invention, compared to native platelets.

FIG. 17 shows generated platelets. The platelets display morphological characteristics identical to ‘native’ platelets. Ultrastructures of generated mouse platelets and control platelets visualized by transmission electron microscopy (TEM). Host platelets were first depleted by intraperitoneal administration of anti-GPIbα antibody R300 (2 µg/g bodyweight) prior to MKs infusion through the heart-lung preparation. Low magnification images are shown for generated platelets in I-III, and control platelets in IV. Detailed images, taken from the low magnification images, are shown in (a)-(d), as indicated. Subcellular structures are shown and annotated as abbreviations: α-G, α-granules; σ-G, σ-granules or dense bodies; Mit, mitochondria; OCS, open canalicular system; MTC, microtubule coils; RBC, red blood cells. Scale bars: 2 µm in I-IV, 500 nm in a-d. Images shown are representative of 4 independent experiments.

### DETAILED DESCRIPTION OF THE INVENTION

The invention provides methods and apparatus as generally described above.

### Example 1 Mouse Ex Vivo Heart-lung Preparation (Representative of ECMO)

Human or mouse megakaryocytes (MKs) were passed through the pulmonary artery in a mouse heart-lung preparation ex vivo. MKs passed all the way through the pulmonary microcirculation and were collected in the system outlet, the pulmonary vein. After multiple re-circulating passages MKs generated large numbers of functional platelets. The results of this approach are shown in FIG. 2.

### Example 2 Assessing Functionality of Platelets Generate Using the Method of Example 1

Mouse platelets were generated by passage of mouse MKs through the ex vivo mouse lung vasculature 18 times. Platelet functionality was assessed by incubation with FITC-conjugated Jon/A antibody which recognises activated integrin a_{IIb}b₃, and platelets then stimulated with thrombin (2 units/ml) or CRP (5 mg/ml) for 90 s. An increase in mean fluoresence intensity was seen upon activation.

### Example 3 Platelet Incorporation Into Growing Thrombus

Microscope views of three levels of focal planes of a representative collagen-induced thrombus, formed under arterial flow rate conditions in vitro, and its cross-section. Generated platelets (yellow and green) occupied all levels of thrombus while host platelets (red) mainly situated on the top of thrombus, suggesting generated platelets may be even more reactive, and functional, in response to collagen, than endogenous platelets.

### Example 4 Tubular Array

The apparatus of the invention comprises multiple tubes, of various of polymer construction and usually up to 200 micron diameter. The tubes are bunched together with spaces of variable width between each of the tubes. Spaces between tubes are usually between 5 and 200 microns. Cells may either be passed through between the tubes, as per arrow (1), or through the tubes, as per arrow (2). The passage of cells may be repeated multiple times. In the configuration where cells are passed between tubes, the tubes themselves may be hollow or solid. The tubes may also be microporous or non-porous.

### Example 5 Platelets Generated Using the Tubular Array in Example 4

Mouse megakaryocytes passaged multiple times through the microporous tubular array generated large numbers of platelets. Mouse megakaryocytes were passaged through the system between tubes, as per arrow (1) in FIG. 5, and samples taken for analysis of platelet numbers each passage. Numbers of passages are indicated on the horizontal axis. Numbers of platelets generated per MK (red line) are indicated on the vertical axis. Platelets were also stained for surface expression of CD42, a marker normally expressed on platelets, and these numbers are also shown (green line). More than 600 platelets were generated per MK.

### Example 6. Platelets Generated From Human Cord Blood Derived MKs Using the Tubular Array in Example 4.

Human megakaryocytes passaged multiple times through the microporous hollow fibre system generate large numbers of platelets. Human megakaryocytes were passaged through the system between tubes, as per yellow arrow in previous figure, and samples taken for analysis of platelet numbers each passage. Numbers of passages are indicated on the horizontal axis. Numbers of platelets generated per are indicated on the vertical axis.

### Example 7. Microchannel Plate

In one embodiment of the invention, the apparatus comprises a barrier defining a number of micro-passages, specifically microchannels. An example of such a barrier is shown in FIG. 8. A plate (usually of glass) is constructed of usually 1-5 cm diameter, 1-5 mm depth, with microchannels (many, often millions) running through the complete plate, each of diameter 1-50 microns. Source material comprising cells is then passed through, repeatedly, as indicated in the diagram. Alternatively, the material can be passed from one side of the barrier to the other and then back again.

### Example 8. Mesh Filter

In one embodiment of the invention, the apparatus comprises a barrier comprising a mesh. An example of such a barrier is shown in FIG. 9. A meshwork (usually of PTFE or other polymer) is constructed. The barrier may be around 1-5 cm diameter. Gaps between fibres (many, often millions) are created by the mesh, each of a width of around 1-50 microns. Source material comprising cells may then passed through the mesh, repeatedly.

### Example 9. Microfluidic System

In an embodiment, the apparatus comprises a microfluidic system. An example of this is shown in FIG. 10. The system comprises a set of channels mimicking a phsysiological vascular system, with a branching structure such that as branching progresssing, the channel diameter decreases, usually by half. The smallest channels are then very numerous (usually 1000 s) and usually 1-15 microns in diameter and depth. Fluid then flows from larger diameter channels (usually up to 100-200 microns) to smaller diameter channels, and then in reverse on the way out of the system. Source material comprising cells is passed through the system, repeatedly. The system may be scaled up, through multiplexing in parallel or series, to allow greater cell volumes to be used.

### Example 10. Functional Platelets Generated From Human CD34+ Derived MKs Passaged Through the Microfluidic Cell.

CD34+derived MKs were passed 6 times through a capilliary mimic microfluidic system. The cells were stained for CD41 and measured by FACS.

### Example 11. Human iPSC-derived Megakaryocytes Generate Functional Platelets in the Microfluidic System.

Human megakaryocytes derived from iPSCs were passaged 6 times through the microfluidic system, and platelets collected, quantified and analysed for functionality by applying to collagen-coated surfaces. In 2 independent experiments, 310 and 406 platelets were generated per megakaryocyte. Platelets generated were able to bind to and become activated by collagen fibres, as shown in the FIG. 12.

### Example 12

Using an ex vivo mouse heart-lung preparation (FIG. 13) the inventors were able to observe the details of platelet generation. Despite their large size, megakaryocytes were readily able to passage through the pulmonary microcirculation, and upon multiple re-circulations generated physiological levels of platelets, 2000-4000 per megakaryocyte. This is the first in vitro system to generate physiological levels of platelets, and also demonstrates that the lung vasculature provides an ideal environment for platelet generation. Megakaryocytes therefore must be able to massively, and reversibly, deform their shape to passage through the capillary network. A defined and reproducible sequence of cell biological changes occurs to megakaryocytes upon repeated passage through the lung vasculature, a process that we now term ‘education’. This sequence begins with the condensation and marginalization of the nucleus, followed by ejection of the nucleus from the cell (enucleation). The process of nuclear condensation continues after enucleation, and the naked nuclei proceed to fragment. After approximately 9 passages through the lung vasculature the anucleate megakaryocytes also begin to fragment to generate preplatelets and eventually platelets, at the rate of 2000-4000 per megakaryocyte by 18 passages through the lung. Finally, dysfunction of this process is likely to play a major role in thrombocytopenias in humans. Mutations in the TPM4 gene, encoding tropomyosin 4, are causal for thrombocytopenias in humans, and here TPM4-/- murine megakaryocytes strikingly generate no platelets in the ex vivo lung preparation, demonstrating not only a critical role for TPM4 in the MK education process, but also clearly showing that platelet generation by lung education is by a different cellular and molecular mechanism to platelet generation in the bone marrow.

Although it is clear that platelets are released from megakaryocytes in an end-stage process for these cells, and that some form of fragmentation of these cells must take place, the location of that process and its details are far from understood. Given the recent demonstration of substantial platelet generation in mouse lung, the inventors chose to develop an ex vivo lung model to understand details of the process. Their first observation was that MKs do not lodge in the microcirculation of the lung, which is initially surprising given their size relative to the internal diameter of capillaries. So, upon first passage of an MK suspension through the vasculature of the lung ex vivo, approximately 70% MKs pass through. This suggests a remarkable ability to deform cell shape and to reform shape upon exit from the microvasculature. Without being bound by theory, this is likely to involve an intimate and dynamic interaction with the endothelium.

This helps to explain an apparent discrepancy between the number of MKs seen histologically in lung tissue, and the argument that they generate substantial numbers of platelets, approximately 50% of total platelet number according to Lefrancais et al. (2017).

Very few MKs are seen in lung tissue histologically, and the inventors have confirmed that in 2-photon sections of labeled MKs, as shown in FIG. 14B. The inventors realised that the fact that MKs passage through the lung vasculature could mean that they may do this multiple times in the in vivo setting, and remarkably when we mimicked this in the ex vivo preparation, MKs were shown to generate large numbers of platelets. These may be seen in the 2-photon images of lung (FIG. 14B) and quantified in the eluate by FACS (FIG. 14C).

A major implication from these observations is that platelet generation may occur anywhere in the vasculature of the body, and that passage through the lung vasculature merely provides the ‘education’ process for MKs as they pass through the lung. The subsequent events of nuclear condensation, marginalization, enucleation and cellular fragmentation to generate platelets may potentially occur at distant sites in the vasculature.

It is, as yet, not clear what is special about the lung vasculature that may provide the MK education process. However, there are differences in the biology of this vessel bed compared to others in the body. One important difference is that the lung vasculature experiences O₂ tension levels higher than elsewhere and CO₂ tension levels lower than elsewhere in the body. These may be important as signals for MK education. The pulmonary vasculature is also under continuous expansion and contraction, as a result of respiration, which may physically promote passage of MKs through the lung vasculature. The inventors addressed the roles of these variables by passaging MKs through lungs normally ventilated, unventilated or ventilated with pure N₂ chronically (1 hour prior to, and for the duration of the MK passage) or acutely (duration of the MK passage only). After 18 recirculated passages through normally ventilated lung, 1x10E4 MKs generated 3x10E7 platelets, as shown in FIG. 14D. Importantly, when lungs were not ventilated, the number of platelets generated per MK dropped to 500, whereas when lungs were ventilated with N₂ acutely, the number of platelets generated per MK was 0. These data indicated that oxygenation was important for platelet generation, but not critical, since platelets were still produced in the absence of O₂. Because there is an additional drop in platelet numbers in unventilated lungs, this indicates that there is a contribution also from the physical forces of ventilation, which may help passage MKs through the microvasculature.

Longer term exposure to N₂, in the chronic setting, is likely however to induce irreversible damage to or death of endothelial cells or severely diminish their viability and functionality. This had a dramatic effect on the ability of passaged MKs to generate platelets, and under these conditions no platelets were generated (FIG. 14D). The lung, which under normal ventilation conditions showed almost no MKs within its structure after MK passage, suggesting that MKs pass through the pulmonary circulation and do not lodge in the microvasculature, by contrast when ventilated with N₂ for one hour before MK passage, showed substantial numbers of MKs ‘stuck’ in the capillary network. This strongly suggests an essential and dynamic conversation between the microvasculature and the MKs, to induce reversible deformation of MKs and stimulate motility through the microvasculature.

Although MKs do not accumulate in the ventilated lung microvasculature, the platelets that they generate do not all pass out of the lung vasculature so efficiently. There is substantial retention of platelets within the capillary network of the lung, and therefore estimation of the numbers of platelets simply from numbers in the eluate, as in the above data, provides a substantial underestimation of the total numbers of platelets generated. To determine the number of platelets retained in the lung microvasculature number, the inventors counted numbers of labeled platelets in lung volume by 2-photon microscopy of fixed lung sections. It was then possible, knowing total lung volume, to estimate the numbers of retained platelets, which are approximately double the number of platelets measured to be released into the eluate. These data are shown in FIG. 14E, where total numbers for the ventilated lung are approximately 3000 per MK, and in the unventilated lung are approximately 1000 per MK.

These are then levels of platelet generation per MK that match estimates of physiological, in vivo, platelet generation. This is a substantial step forward in ex vivo synthesis of platelets, being approximately an order of magnitude greater than currently published methods for platelet generation outside the body. We have estimated these numbers based upon solely the numbers of platelets labeled with anti-CD41-FITC dye. This is because we know that these platelets are generated from the MKs that had been stained in vitro with anti-CD41-FITC prior to passage through the lung. So, all calculations above related to those labeled platelets. In addition to labeled platelets, however, FACS analysis identifies significant numbers of unlabeled platelets. These are likely to derive from two sources. As the inventors show, a substantial number of platelets only slowly traverse through the lung microvasculature, and therefore platelets are ‘retained’ in the lung vasculature even after multiple fluid passages. There will therefore be likely to be a pool of unlabeled endogenous host platelets in the lung. In addition, Looney et al. demonstrated that the mouse lung is a site of extravascular MKs, which may also be generating numbers of platelets, which will be unlabeled.

In addition to the physiological levels of platelets generated in the ex vivo lung system, another major step forward with this approach is that the platelets generated are fully and normally functional. FIG. 15 shows functionality in both the labeled (donor-derived) and unlabeled (host-derived) platelets. The latter form a valuable control for the functionality of the stained, generated platelets. Both donor- and host-derived platelets respond with activation of integrin α _{IIb}β ₃ and surface expression of P selectin (a measure of α-granule secretion) in response both to thrombin or collagen-related peptide (CRP). Although not achieving significance, there is a consistent trend towards greater functionality in donor-derived platelets than host-derived platelets. One explanation may be that all donor-derived platelets are very young, having only just been generated, whereas the cellular age of the host-derived platelets will span from very young through to old, senescent platelets, so their mean age will be considerably greater. It has been reported previously that young platelets are more responsive than older platelets, and this may explain the greater responsiveness in the donor-derived cells. Interestingly also, although generating platelets under ‘no ventilation’ conditions leads to fewer platelets per MK, their functionality is generally comparable to those derived under normal ventilation conditions (FIG. 15B). Donor-derived platelets also behave normally in formation of thrombi. FIG. 15C shows labeled donor-derived platelets, which when mixed into recipient mouse blood, behave in a manner similar to their unlabeled host cells, and become incorporated fully into thrombi that form under flow on collagen-coated surfaces. Interestingly however, donor platelets were consistently early interactors with collagen, situated close to the exposed collagen fibres, presumably reflecting again a marginally greater functionality of these younger platelets.

The ex vivo lung system has also allowed the inventors to determine the mechanism underlying the transformation of MKs to platelets, and observe the changes to MKs upon repeated passage through the pulmonary vasculature. The most striking finding is that MKs enucleate within the vasculature, in a step preceding fragmentation to generate platelets within the blood stream. FIG. 16 shows the steps involved in the process, with images shown in FIG. 16A, quantified in FIG. 16B and summarized diagrammatically in FIG. 16C. During the low passage numbers, the large polyploid nucleus moves from a central position to the periphery of the cell, in a process of marginalization. The nucleus is then extruded from the cell upon further passages through the lung vasculature, until by approximately 9 passages there are very few nucleated MKs left. At this point the naked nucleus, extruded from the cell, undergoes a process of division into multiple component sub-nuclei, which proceed to condense into small, compacted sub-nuclear units. The anucleate MK proceeds to fragment upon multiple further passages through the lung vasculature, reaching plateau numbers of platelets by 15-18 passages (see FIG. 13D).

Finally, we were interested to see whether MKs from a genetically modified mouse with macrothrombocytopenia, lacking expression of tropomyosin 4 (TPM4), would be defective in platelet generation in the ex vivo lung system. TPM4 mutations are associated with a rare form of macrothombocytopenia in humans, where the platelet count drops to about 50% of normal. Similarly, platelet counts in TPM4-/- mice drop by approximately the same amount compared to wild types. TPM4-/- MKs culture in vitro normally, but when passaged through ex vivo mouse lung, these cells remarkably generate almost no platelets. This striking result suggested that TPM4, which regulates the actin cytoskeleton, is absolutely required for all platelet generation by the lung education mechanism. We observed however that normal numbers of TPM4-/- MKs passage through the lung, and so TPM4 is clearly not required for reversible deformation of these cells, but rather for one of the subsequent steps. These observations explain the reduction in platelet count to 50%, because Lefrancais et al. had proposed that approximately 50% of platelet generation takes place in the lung vasculature, and the other 50% in the bone marrow. Additionally, and importantly, the data demonstrate clearly that the mechanism for platelet generation in the lung vasculature is fundamentally different from that in the marrow. The inference is that TPM4 is likely not to be required for platelet generation in the marrow, but that it is required for the processes of platelet formation in the lung vasculature. There are therefore likely to be at least 2 distinct mechanisms for generation of platelets in the body, one of which is lung education which accounts for 50% of platelet generation in the mouse. Clearly it will be important to determine whether this is the case for humans, and if so, what proportion of platelets are generated by lung education. It also raises the possibility that there are functionally different populations of platelets in vivo, because they are generated by distinct molecular and cellular mechanisms. For example, we know that only a proportion of platelets are capable of undergoing a procoagulant response, exposing phosphatidylserine on the platelet surface (Agbani & Poole, 2017). It will be important to determine whether this is the product of different generation mechanisms for different platelet populations.

Regarding TPM4, it is now clear that we can conclude the following:

TPM4 has a role to play in the structure/formation of multi-lobed nuclei in MKs, since their morphology differs in the TPM4-/- cells.

TPM4 is not essential for passage of MKs through the lung vasculature. It may have a partial role in cell motility here, but it is not essential, since cells get through.

TPM4 is also not essential for enucleation, since there are many naked nuclei in the lung sections, and also in the eluate after lung passage.

However, TPM4 is likely to be critical for the final fragmentation event where the MK ghosts break into platelets, since the ghosts are present in the eluate after multiple passages through lung, but the platelets are absent.

Example 13. Sizing of platelets produced.

Platelets were produced using the method of the invention. They were stained, and then combined with native platelets. The size of the native platelets and the platelets produced using the method of the invention were measured and compared. The results are shown in FIG. 16. As can be seen, the platelets produced were significantly larger than native platelets and were generally similar in size to each other.

1. A method for generating platelets or platelet-like particles comprising the step of:
a) passing a source material comprising one or more cells through a micro-passage to produce a passaged material, the passaged material preferably comprising platelets or platelet-like particles.

2. The method of claim 1, wherein the cells are selected from stem cells, particularly iPSCs; intermediate and/or final products of stem cell differentiation such as hemogenic endothelia, hematopoietic progenitor cells, megakaryocytes, endothelial cells, leukocytes, erythrocytes bone marrow cells, blood cells, lung cells and cells comprising basement membranes.

3. The method of claim 1 or claim 2, wherein the cells are megakaryocytes or precursors thereof.

4. The method of any preceding claim, wherein the step of passing the source material through the micro-passage particularly comprises or consists of passing one or more, especially a plurality of cells, through one or more micro-passages, especially through a plurality of micro-passages.

5. The method of any preceding claim, wherein the step of passing the source material through a micro-passage comprises passing the source material from a first region, through the micro-passage to a second region.

6. The method of claim 5, further comprising the step of applying pressure to the source material in the first region to drive the source material to the second region.

7. The method of any preceding claim, wherein the step of passing the source material through a micro-passage comprises passing the source material through a plurality of micro-passages.

8. The method of any preceding claim, wherein the micro-passage is generally less than 5 mm in length.

9. The method of any preceding claim, wherein at least one of the width and height of the micro-passage is between 1 and 250 µm, between 1 and 200 µm, between 1 and 150 µm, between 1 and 100 µm, between 1 and 50 µm, between 1 and 25 µm or between 1 and 10 µm at a point along its length.

10. The method of claim 9, wherein the height and/or width of the micro-passage is less than 250 µm, less than 200 µm, less than 150 µm, less than 100 µm, less than 75 µm, less than 50 µm, less than 25 µm or less than 10 µm at at least one point along the length of the micro-passage.

11. The method of claim 9 or claim 10, wherein the width and/or height of the micro-passage is more than 1 µm, more than 5 µm, or more than 10 µm at at least one point along the length of the micro-passage.

12. The method of any preceding claim, wherein the method comprises passing the material through a micro-passage more than once.

13. A method according to any preceding claim, wherein the step of passing a source material comprising cells through a micro-passage comprises the following steps:
a) providing a source material in a first vessel;
b) passing the source material from the first vessel into a second vessel, via one or more micro-passages to produce a passaged material.

14. A method according to claim 13, wherein the method further comprises the step of
c) passing the passaged material from the second vessel into the first vessel, via one or more micro-passages.

15. A method according to claim 14 or 15, comprising repeating steps b) and / or c).

16. A method according to any preceding claim, further comprising the step of analysing the passaged material for the presence of platelet or platelet-like particles.

17. A method according to any preceding claim, further comprising the step of extracting platelets or platelet-like particles from the passaged material.

18. A method according to any preceding claim, wherein the step of passing the source material or the passaged material through a micro-passage comprises passing the material through ECMO (Extra Corporeal Membrane Oxygenation) machine; a flow cell, particularly a high pressure flow cell; a barrier comprising multiple microchannels, for example a microchannel plate; a mesh filter; or a tubular array.

19. A apparatus for producing platelets, the apparatus comprising a first region in which a source material may be provided and a second region for receiving a passaged material, the first and second regions being connected by at least one micro-passage.

20. The apparatus according to claim 19, comprising a means for driving the source material from the first region to the second region.

21. The apparatus according to claim 19 or 20, wherein the micro-passage is generally less than 5 mm in length.

22. The apparatus according to claim 19, 20 or 21, wherein at least one of the width and height of the micro-passage is between 1 and 250 µm, between 1 and 200 µm, between 1 and 150 µm, between 1 and 100 µm or between 1 and 50 µm at a point along its length.

23. The apparatus of any of claims 19 to 22, wherein the height and/or width of the micro-passage is less than 250 µm, less than 200 µm, less than 150 µm, less than 100 µm, less than 75 µm, or less than 50 µm at at least one point along the length of the micro-passage.

24. The apparatus of any of claims 19 to 23, wherein the width and / or height of the micro-passage is more than 1 µm, more than 5 µm, or more than 10 µm at at least one point along the length of the micro-passage.

25. The apparatus of any of claims 19 to 24, wherein the first and second regions are generally connected by at least 100, at least 200, at least 300, at least 400, at least 500, at least 1,000, at least 5,000, at least 10,000, at least 100,000, at least 250,000, at least 500,000, or around or at least 1,000,000 micro-passages.

26. The apparatus of any of claims 19 to 25, wherein the micro-passages are arranged in a barrier or membrane, positioned between the first and second regions; or in a tubular array.

27. The apparatus of any of claims 19 to 26, further comprising a means for returning the passaged material from the second region to the first region.

28. The apparatus of any of claims 19 to 27, further comprising a means for counting cells or particles and/or sorting cells or particles by size.

29. Platelets or platelet-like particles, obtained using the method of the invention.

30. The platelets or platelet-like particles according to claim 29, wherein the platelets are substantially homogenous in age and/or size.

31. A pharmaceutical composition comprising platelets or platelet-like particles according claim 29 or 30.

32. A method of treating a subject in need of platelets or platelet-like particles comprising the step of administering the platelets or platelet-like particles according to claim 29 or 30 or one or more extracts therefrom to the subject.

33. A platelet or platelet-like particle according to claim 29 or 30, for use in therapy, particularly in the treatment of a subject in need of platelets or platelet-like factors.
